(19) United States
(12) Patent Application Publication
(10) Pub. No.: US 2005/0043659 A1
(43) Pub. Date: Feb. 24, 2005

(54) Apparatus and method for treatment of long bone fractures

(76) Inventors: Murray John Challis, Verrierdale (AU); Terence John Hickey, Mount Elisa (AU)

Correspondence Address: OHLANDT, GREELEY, RUGGIERO & PERLE, LLP, ONE LANDMARK SQUARE, 10TH FLOOR, STAMFORD, CT, 06901, US

(21) Appl. No.: 10/489,504

(22) PCT Filed: Sep. 18, 2002
(86) PCT No.: PCT/AU02/01279

(30) Foreign Application Priority Data
Sep. 18, 2001 (AU) PR 7739

(51) Int. Cl. A61F 5/00

(52) U.S. Cl. 602/5

(57) ABSTRACT

An apparatus and method for treating a long bone fracture in the limb of an animal wherein a non-invasive technique is provided for intermittently inducing a shortening in length of the skeletal musculature surrounding the long bone, whereby a compressive force may be intermittently applied to the fractured bone. Suitably a radial inward force is applied substantially fully around the circumference to the limb, by an inflatable cuff or sleeve, that is transmitted to the surrounding muscles. Alternatively, electrical signals cause active contraction of the musculature. The treatment may include the application of about 55 to 65 compressions each day for at least 10 days, and commencing within about 7 days of the fracture being sustained. The compressions, applied with frequency in the range of 0.05 Hz to 2 Hz, are desirably just short of that which produces fracture site pain and the intensity of the compressions may be increased between treatment sessions, consistent with heating.

### BACKGROUND OF THE INVENTION

1. Field of the Invention

This invention relates to a method of treating a long bone fracture in a warm-blooded animal, particularly a human being, in order to promote the healing of the fracture. The invention also extends to an apparatus for use in promoting the healing of long bone fractures and a compression device for use with the apparatus.

This invention relates particularly, but not exclusively, to a method and apparatus for treating fractures of the distal radius, i.e. of the forearm. It will therefore be convenient to hereinafter describe the invention with reference to this example application. It is to be clearly understood however that the invention is capable of broader application, for example it might be applied to leg bones and also other long bones.

2. Discussion of the Background Art

Long bone fractures are fairly common injuries for animals, including human beings. They typically result from externally applied trauma as a result of accidents, such as falls, motor vehicle accidents, sporting injuries or the like. Typical long bone fractures include tibia and fibula fractures of the lower leg, femur fractures of the upper leg and humerus, ulna and radius fractures of the arm.

The established clinical treatment of these fractures involves immobilising the fractured limb, e.g. by means of a plastic or a plaster cast, until such time as the bone has knitted at the fracture site. Thereafter the patient is treated with physiotherapy to restore limb function, strength and range of movement. The latter is particularly important as immobilisation results in some muscle atrophy and joint stiffness and muscle tone needs to be restored. Where some displacement of the fractured bone has occurred it is also necessary to position the opposed ends of the fractured bone in face to face abutment before the limb is immobilised.

The time taken for the healing of a fracture naturally varies from case to case. A particularly important factor is the mechanical environment of the fracture site. Fracture healing time also depends on factors such as the severity of the fracture, the age of the patient, and the bone physiology of the patient. It is also known in patients with long bone fractures, particularly those sustained to the leg bones, that the reintroduction of normal mechanical stimulus to the fracture, such as by weight bearing during assisted walking, can improve bone healing: see Goodship, A. E. & Kenwright, J.; “The influence of induced micromovement upon the healing of experimental tibial fractures”, J. Bone and Joint Surg., 67-B(4):650-655, 1985.

Naturally the time taken for a patient to restore function and range of movement can be very important to a patient in the modern world. Many people rely on the use of their arms and hands to earn a living, for example sportspeople, manual labourers, brick layers or typists to name a few. These people would generally want a fracture to heal as quickly as possible. Clearly therefore any development that was able to accelerate the repair and healing of long bone fractures, particularly common fractures such as fractures of the distal radius, would be commercially very important.

It is known in the art to use an external fixation device to hold fractured bone/s in position during a healing process. This treatment tends to be used on more complex fractures. An external fixation device generally comprises broadly attachment elements directly attached to the bone on either side of the fracture and projecting laterally outwardly through the skin of a patient. The attachment elements are then attached to each other by at least one, typically two, longitudinal tensioning elements. The longitudinal elements apply a longitudinal compressive force to the fractured bones to hold the bones together for correct knitting of the fracture. An external fixation device is invasive passing through the skin and flesh of the patient and being directly attached to the bone of a user.

It has been known to vary the compressive force applied to the bone having the fracture using the external fixation device. This typically involves manual adjustment of the longitudinal tensioning element. It is also known that the dynamic loading of long bone fractures using an external fixation device can increase the rate of healing of a long bone fracture, such as described for example in U.S. Pat. No. 5,997,490 (McLeod et al) which involves a cyclical stimulation having a frequency of between about 5 Hz and about 20 kHz. This specification also happens to contain a useful review of the related prior art. However it is not really practical to apply an external fixation device to a fracture site simply to get some speeding up of the healing process. The treatment is simply too complex and too invasive.

However, it would clearly be advantageous if a non-invasive apparatus and method could be devised for taking advantage of this discovery, namely that dynamic loading of the fracture site tends to speed up the healing of long bone fractures. Such a method and apparatus would enable this discovery to be conveniently and widely used to accelerate healing of long bone fractures and thereby improve quality of life for patients.

### SUMMARY OF THE INVENTION

Object of the Invention

It is therefore an object of the present invention to provide a practical, non-invasive apparatus and corresponding method for the dynamic loading of long bone fractures to enhance or accelerate healing of many commonplace long bone fractures, such as fractures of the distal radius.

Other objects of the present invention will be apparent from consideration of the following description.

Disclosure of the Invention

According to an aspect of this invention there is provided a method for treating a long bone fracture in a limb of an animal or a human, the method including the steps of:

non-invasively and intermittently inducing a shortening in the axial length of skeletal musculature surrounding the fractured long bone in said limb;

whereby, the musculature intermittently applies a longitudinal compressive force to the fractured bone.

The step of inducing of shortening in the length of the musculature can be achieved non-invasively by either causing the muscles to contract by a stimulus, such as an electrical stimulus or else by applying a radial force to the outside of the limb that is transmitted to the muscles to cause a shortening in the longitudinal length thereof.

The force or stimulus may be dynamic in the sense that it varies as a function of time. Preferably the force is applied at regular intervals and in a balanced fashion.

In one form the force may be applied by a force transmitting member that extends fully circumferentially around the limb, and the force may be applied substantially fully radially inwardly around the circumference of the limb.

The forces may be applied to the fractured limb to effect a plurality of compressions of the long bone over a predetermined period of time, for example in a treatment session.

The method may include applying at least 20 compressions in a treatment session, preferably 40 to 70 compressions, more preferably about 55 to 65 compressions in a session.

The compressions suitably involve application of a force having a magnitude below that which causes the patient pain at the fracture site, suitably a magnitude just below that which causes pain or at most moderate discomfort. Further the compressions may all be of substantially the same magnitude so as to apply the same force to the surface of the limb of the patient each time it is applied. Further each compression may last 2-7 seconds, preferably 3-4 seconds.

In another form of the present invention, the shortening in the length of skeletal musculature surrounding the fractured long bone is induced by an electrical stimulus provided by an electrical signal transmitter. The muscles may be stimulated with the same frequency and intensity as described above with reference to the compressions.

Suitably as healing takes place, the electrical stimulus or mechanical forces might be increased in amplitude every few days or every week up to the point that falls just short of producing fracture site pain or at most moderate discomfort. The method may include at least one treatment session as described above each day, preferably two treatment sessions each day spaced 8 to 16 hours apart, e.g. 10 to 14 hours apart. Preferably the method includes conducting the treatment sessions for at least 10 days, preferably at least 20 days and more preferably for at least 28 days, depending on the particular fracture. Typically the treatment sessions might commence about 7 days post fracture, and preferably within 5 to 10 days.

According to another aspect of this invention there is provided an apparatus for treating a long bone fracture in the limb of an animal or human, said apparatus including:

non-invasive means for inducing a shortening in the axial length of the skeletal musculature surrounding the long bone, whereby a compressive force may be intermittently applied to the fractured bone.

In one form of the apparatus, known as the fracture cuff form, the means for inducing a shortening of the skeletal musculature may comprise a force application member applying a force to the limb of the patient. This external application of force causes a shortening in the longitudinal length of the musculature.

Typically the force is applied to the surface of the skin of the patient, i.e. in a radial inward direction, and the force application member extends fully around the limb of the patient, and suitably applies a force fully circumferentially around the limb of the patient.

The force application member need not apply the force fully around the circumference of the limb of a user. It is however highly desirable that the force is balanced, for example by diametrically opposed radially inwardly directed forces that balance with one another. Thus the apparatus applies pressure externally to the fractured limb of a patient, i.e. non-invasively, to cause a shortening in the longitudinal length of the skeletal musculature which in turn applies compression to the fractured bone.

Further the force application member most suitably applies the force dynamically, for example intermittently with a frequency in the range of 0.05 Hz to 2 Hz, preferably from about 0.1 Hz to 1 Hz. That is, the pressure is applied for a short period and then released by the force application member, and thereafter this cycle of force application and release may be repeated.

The force application member may be a sleeve or cuff or bladder that passes over the limb of a user and that is expandable and contractible on demand to apply said force to the limb of the patient. Any material or means may be used to expand and contract the cuff and it need not be a fluid. Conveniently the sleeve or cuff is inflated with a gas, for example air.

An inflatable sleeve or cuff typically having a length of 10-25 cm may be formed by joining two superimposed sheets joined together around the edges. It typically has a flattened shape when in a deflated condition. Typically the sleeve or cuff is desirably formed in a ring or toroidal shape, being continuous in the sense that it does not have means for opening the sleeve at a point. It may be fitted to a patient by passing it through the hand of the patient and over the arm.

In a particularly preferred form, the force application member may be inflated and deflated on demand by the forced introduction and subsequent release of air, for example through a valve. While it is practical and convenient to use air, it is to be appreciated that any other form of fluid may also be used.

Thus compressed air may be used to inflate the sleeve or cuff and apply a force in the form of cuff pressure to the limb, for example the arm of a patient. Thereafter the air may be vented to deflate the sleeve and relieve the pressure.

Typically the apparatus also includes an immobilisation means for immobilising the fractured limb of the patient. In one form the immobilisation means comprises a rigid cast of plastic or plaster.

The force application member, for example the cuff, may be positioned under the immobilisation member, i.e. sandwiched between the immobilisation member and the skin of the patient. Alternatively, the force application member may be adjacent the immobilisation member, preferably abutting the proximal end of the immobilisation member. Conveniently the force application member may be attached to the immobilisation member, although this is not necessary.

When the force application member is an inflatable cuff, it may incorporate a valve for selectively inflating and deflating the cuff and the valve may project through the immobilisation member to the outside where it can easily be coupled to an inflation device, for example a pump. Alternatively, the valve may be remote from the cuff which includes a coupling projecting through the immobilisation member. This may provide one form of attachment of the cuff to the plaster cast in one form.

The apparatus may further include a means for selectively inflating the cuff with compressed air to a predetermined pressure for a predetermined time and then deflating the cuff. Furthermore, the inflating means may be controlled by timing means.

In another form of the apparatus, called the electrical impulse form, the means for inducing a shortening in the axial length of the skeletal musculature comprises an electrical signal transmitter for generating an electrical impulse for stimulating the skeletal musculature and thereby causing it to contract and shorten to apply longitudinal compression to the bone.

The electrical signal transmitter may comprise a signal transmitting member that is placed in contact with the outer surface of the limb of the patient and applies an electrical impulse to the limb of the patient. Typically the member might be an electrode placed on the skin of a user. The electrode may be arranged to pass fully circumferentially around the arm of a user so as to induce contraction of muscles in the limb at the same time. This arrangement helps to achieve simultaneous coordinated contraction of the musculature, including those muscles traversing the fracture.

The electrical signal transmitting member may deliver an electrical impulse that resembles that delivered by a transcutaneous electrical nerve stimulation (TENS) apparatus used in physiotherapy, or more particularly that delivered by a high voltage pulsed current therapeutic device similar to that used for rehabilitation of weak muscles. The TENS apparatus has a small silver plated electrode that is placed on the skin of the patient.

The electrical impulses cause contraction of the skeletal musculature of the fractured limb which applies a longitudinal compression to the bone. The electrical impulses are applied intermittently as described above for the fracture cuff form of the invention. The signal transmitting member, for example electrode, may be electrically coupled to a signal generating means of a type that is well known in the art. Such a generator would typically be portable, be battery powered and suitability utilise digital logic circuits to allow desired stimulus to be programmed as required.

The electrical stimulus comprises an underlying carrier signal that is amplitude modulated by an impulse signal. The carrier signal preferably has a frequency in the range from about 30 Hz to 50 Hz, whilst the modulating impulse signal has a frequency in the range of 0.05 Hz to 2 Hz, most suitably from about 0.1 Hz to 1 Hz.

According to a third aspect of this invention there is provided a compression apparatus for inflating and deflating the cuff or sleeve (in the fracture cuff form of the apparatus), the apparatus pressurising and inflating the cuff by supplying compressed air to the cuff and then also deflating the cuff after it has been inflated, such that the cuff is intermittently inflated and deflated, for example on a regular basis.

Thus the compression apparatus comprises means for inflating a cuff such that it applies pressure radially inwardly to the limb, for example arm, of the user and then, after the desired compression of the fracture has been achieved, relieves that pressure and deflates the cuff and then thereafter compresses the sleeve again, etc.

The compression means may comprise a closed tank container, a valve means having an air inlet port operatively coupled to the pressurised air supply and a vent port for venting air to the atmosphere in parallel with the compressed air inlet, and a cuff coupling for operatively coupling the valve means to the cuff, the valve means having a switch means for switching either the compressed air inlet port or the vent port to the cuff at any one time.

Thus only one of the compressed air inlet or the vent for compressed air can be coupled to the bladder or cuff at any one time. Thus the apparatus is either in compression mode or deflation mode at any one time. Conveniently the valve means may comprise a solenoid actuated valve, for example a 240 volt solenoid valve.

The pressurised air supply may comprise a pressurised tank, for example a cylinder of air.

The apparatus may also include a compressor in communication with the closed tank, and means for isolating the compressor from the tank once sufficient compressed air has been pumped into the tank. Thus the compression apparatus permits the amount of air that is pumped into the tank to be varied before it is isolated.

Once the tank has been isolated from the compressor, the valve means opens placing the tank in communication with the cuff and allowing the cuff to be filled with compressed air. The pressure generated in the cuff depends on the amount of compressed air admitted to the cuff from the storage tank, which can be regulated as desired.

The compressor apparatus may further include timer setting means for setting the time for which the cuff is inflated (inflation time) and also the time for which the cuff is deflated (deflation time). Typically the switch means associated with the valve means switches between the compressed air inlet port and the vent port based purely on time, for example a predetermined time interval.

Typically the apparatus also includes typical safety features such as a pressure cut-out switch to prevent over-pressurising the storage tank.

### BRIEF DETAILS OF THE DRAWINGS

An apparatus and a method in accordance with this invention may manifest itself in a variety of forms or embodiments. It will be convenient to hereinafter describe in detail one preferred embodiment of the invention with reference to the accompanying drawings. It is to be clearly understood however that the detailed nature of this specific description does not supersede the generality of the preceding broad description outlining the general principles of the invention. In the accompanying drawings:

FIG. 1 is a schematic sectional side view of apparatus in accordance with one embodiment of the invention, namely a fracture cuff, mounted on a patient's arm for treating a fracture of the distal radius or wrist;

FIG. 2 is a side view of the fracture cuff of FIG. 1, showing the application of pressure to the arm of the patient;

FIG. 3 is a schematic circuit diagram of a compression apparatus used with the apparatus of FIG. 1;

FIG. 4 is a graph showing pressure as a function of time during the typical treatment session;

FIG. 5A is a graph plotting recovery in grip strength as a function of time after a fracture using the invention compared with standard treatment;

FIG. 5B is a graph plotting recovery in pinch strength as a function of time after a fracture using the invention compared with standard treatment;

FIG. 5C is a graph plotting recovery in key grip strength as a function of time after a fracture using the invention compared with standard treatment;

FIG. 5D is a graph plotting recovery in isometric supination strength as a function of time after a fracture using the invention compared with standard treatment;

FIG. 6 is a schematic sectional side view of an apparatus in accordance with another embodiment of the invention, which employs electrical impulses;

FIG. 7 is a graph depicting the electrical stimulation signal as a function of time during the typical treatment session; and

FIG. 8 is a graph showing the compression force at a fracture site resulting from use of the embodiment of the invention described in relation to FIGS. 1 to 4 above.

### DESCRIPTION OF EMBODIMENTS OF THE INVENTION

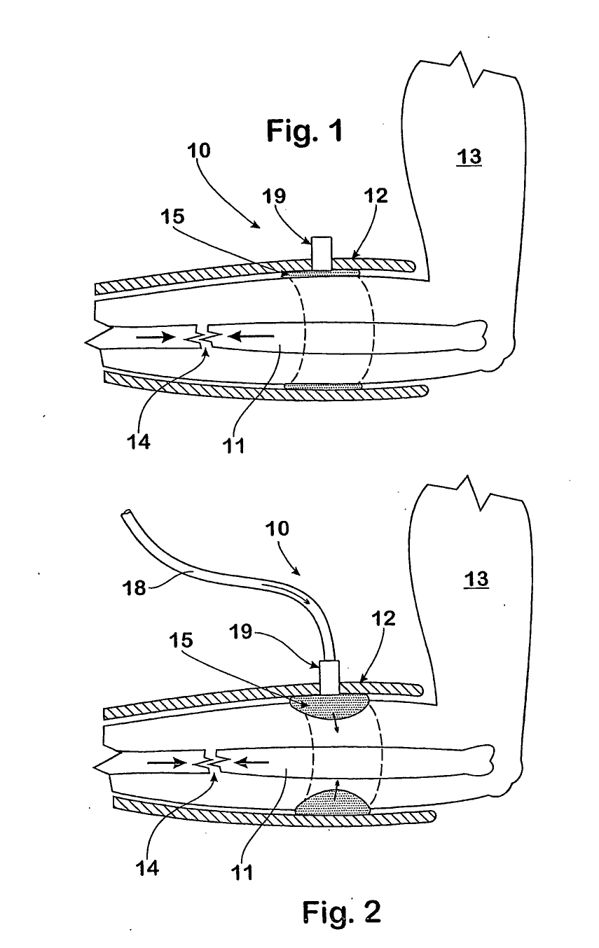

In FIGS. 1 and 2, reference numeral 10 refers generally to an apparatus in accordance with a first embodiment of the invention. The apparatus 10 includes an immobilisation cast 12 for a fracture 14 of the distal radius 11 of a patient. The cast 12 is fitted around the fractured arm 13 of the patient and a force application member 15 is sandwiched between the immobilisation cast 12 and the outer surface or skin of arm 13.

The immobilisation cast 12 may be in the form of a moulded plastic cast or else in the form of a well known plaster cast that is shaped to fit the arm of the patient. The structure and function of the immobilisation cast 12 would be well known to persons skilled in the art and will not be described in further detail in this specification.

The force application member 15 in the illustrated embodiment comprises an inflatable cuff or sleeve or bladder that extends circumferentially around the arm of the patient. The cuff 15 is similar to an inflatable armband used to assist flotation of infants in swimming pools. It is also similar to the cuffs used to constrict the arm in blood pressure measurement devices, such as a sphygmanometer, although the cuff of the embodiment has a ring or toroidal type configuration.

The inflatable cuff 15 has a coupling 19 protruding through the immobilisation cast 12 for passing compressed air into the cuff 15 and also for venting compressed air from the cuff 15, as required. The coupling 19 is provided for coupling the cuff 15 via a tube 18 to a valve of a compression apparatus, which will be described in more detail below.

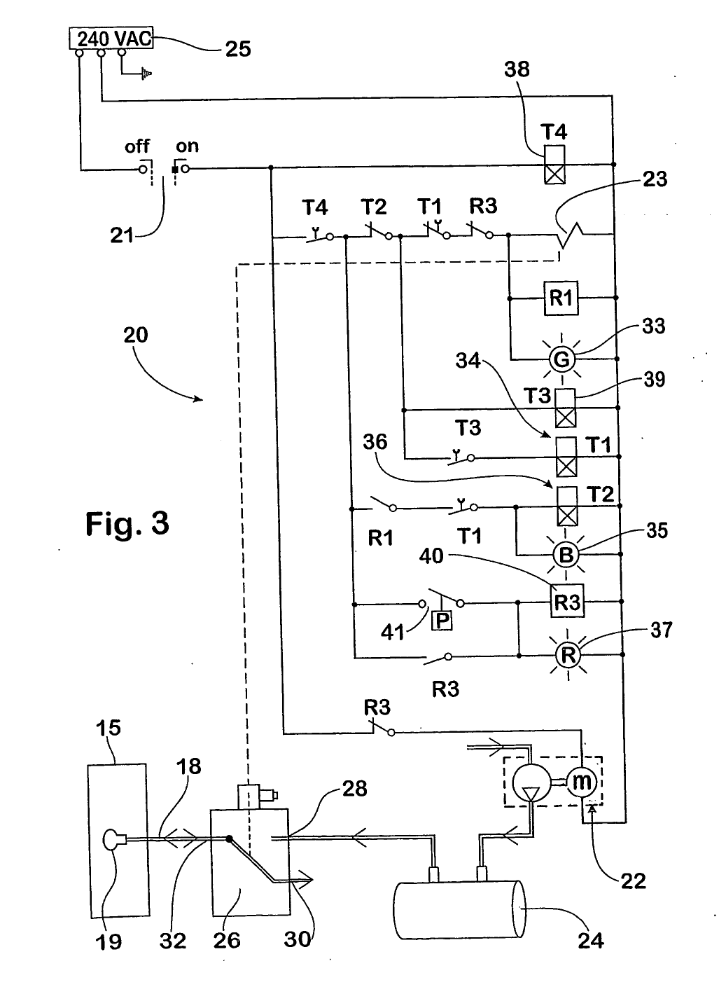

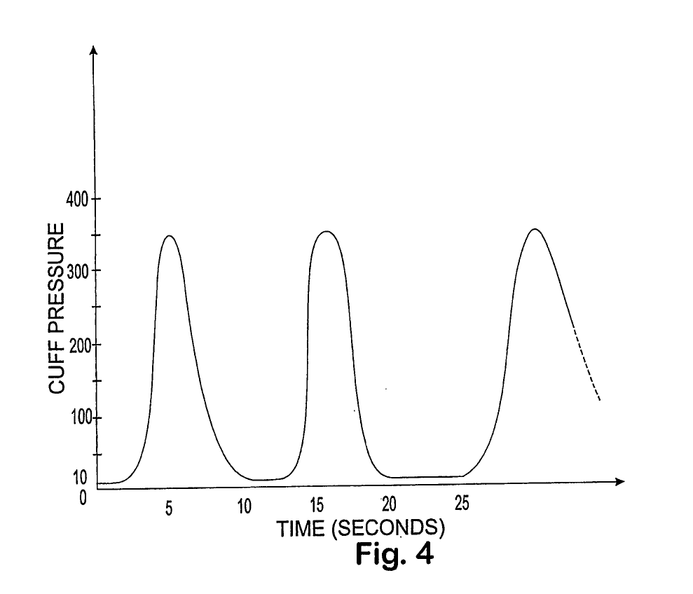

FIG. 3 is a schematic circuit diagram for a compression apparatus 20 to be used with the cuff 15 shown in FIGS. 1 and 2. Broadly, the apparatus 20 comprises a fluid circuit having an electric motor (m) driven air compressor or air pump 22 in fluid communication with an air tank 24 in the form of a stainless steel cylinder. The compressor is electrically powered from a 240v AC supply 25 through a main switch 21. The compressor 22 can be isolated from the tank 24 once a predetermined quantity of compressed air has been pumped into the tank, as will be described in more detail below.

The air tank 24, in turn, is in fluid communication with a valve in the form of a solenoid valve 26. The solenoid valve 26 has two ports on one side, namely an air inlet port 28 and a vent port 30. The air inlet port 28 is coupled to the air tank 24 and the vent port 30 is in fluid communication with an air vent to atmosphere. The valve 26 has a third port 32 on the other side that is in fluid communication with the cuff 15 via the tube 18 attached to the coupling 19. The valve 26, which is electrically controlled by solenoid winding 23, has the ability to switch between placing the air tank 24 in communication with the cuff 15 through air inlet port 28 and placing vent port 30 in communication with the cuff 15.

The compression apparatus 20 also includes an electrical circuit having an inflation timer (T1) 34 for setting the time for which the air tank 24 is coupled to the cuff. The apparatus further includes a deflation timer (T2) 36 for determining the time for which the vent port 30 is coupled to the cuff 15. Each of these timers 34, 36 is continuously variable and can be individually set by a user who thus has control over the times, and thereby also the inflation and deflation cycle of the cuff 15.

The circuit diagram in FIG. 3 shows the inflation and deflation timers (T1, T2) and respective indicators, including an inflate indicator 33 in the form of a green LED (G), a deflate indicator 35 in the form of a blue LED (B) and a high pressure indicator in the form of a red LED (R) 37. The circuit for the compression apparatus 20 also includes a start-up timer (T4) 38 and a transition timer (T3) 39. The apparatus further includes a high-pressure lockout relay (R3) 40 actuated by a pressure switch (P) 41 coupled to the air tank 24 for shutting down the apparatus 20 if a predetermined air pressure is exceeded. This is a safety feature. The structure and function of these features would be well known to a person skilled in the art and accordingly will not be described in further detail in the specification.

In use, the apparatus 20 is started up with the on/off switch 21 and the compressor 22 starts to produce compressed air. The inflation or compression stage is initiated by pumping a certain volume of air into the tank 24 with the compressor 22. The amount of air pumped into the tank 24 and thereby the air pressure in the tank can be set as desired by a user. The tank 24 is then isolated from the compressor and the valve 26 switched to allow the air in the tank 24 to flow into the cuff 15 and inflate the cuff. After the inflation time has elapsed, the valve 26 switches to put the cuff or bladder 15 in communication with the vent port 30. This permits air to vent from the cuff 15 to atmosphere. Again the deflation time can be similarly set by a user by way of timer 36. These time settings also enable the total duration and thereby the frequency of the cycles to be adjusted. For example in one application, the inflation stage may be set at 3.5 seconds and the deflation stage at 6.5 seconds to give a total cycle time of 10 seconds and 6 cycles per minute.

In use the apparatus 10 as a whole is used to treat a fractured limb, a fracture 14 of the distal radius 11 in the example, in a number of treatment sessions to assist, enhance and/or accelerate healing of the limb. The cuff 15 and specifically the coupling 19 thereof is first coupled to the compression apparatus 20 that supplies the compressed air via solenoid valve 26, and then the apparatus 20 is switched on. This causes the cuff 15 to inflate for a period of a few seconds, e.g. about 3.5 seconds to a pressure where it stops short of inflicting any fracture pain or at most moderate discomfort and then deflates releasing the grip on the patient's arm 13. This grip and release of the arm 13 by the cuff 15 in the present embodiment causes and/or simulates co-contraction of the forearm skeletal musculature (not shown) and applies a longitudinal compression force to the fracture 14.

This sequence of application of pressure to a circumferential portion of the forearm for about 3.5 seconds and then relieving the pressure for about 6.5 seconds constitutes the compression cycle. The compression apparatus 20 then automatically repeats this cycle over about 10 minutes, effecting about 60 compressions. Thereafter the compressed air is vented from the cuff 15 and the pressure is relieved from the forearm entirely.

The cuff or sleeve 15 is desirably inflated with compressed air to the point where it applies a firm pressure, just short of inflicting fracture site pain, radially inwardly against the flesh of the arm of the patient, and maintains this pressure for about 3.5 seconds. The duration of each compression is substantially the same. Further the intensity or strength of the force applied by the cuff against the arm of a user is substantially the same for each compression, as illustrated by the curve shown in FIG. 4. The carrying out of the treatment to a patient generally does not cause any pain or at most moderate discomfort.

The treatment sessions are suitably carried out twice a day over about 4 to 6 weeks for fractures of the distal radius. The treatment sessions may be carried out by the patients in their own home without the assistance of a treatment specialist. The applicant has conducted experiments on patients with fractures of the distal radius to determine the effectiveness of the apparatus of the preferred embodiment.

During these experiments, several patients had the fracture cuff fitted within 5 to 14 days of the fracture being sustained, with the average time being 9.4 days post fracture. The fracture cuff comprised a normal plaster cast 12 fitted over the patient's forearm with an inflatable cuff or sleeve 15 sandwiched between the cast and the arm 13 in the region of the fracture 14. For a fracture to the distal radius, the inflatable cuff or sleeve 15 is suitably fitted to the proximal third of the forearm musculature or approximately one third (⅓) of the way along the limb from the patient's elbow. The patients were then subject to two treatment sessions of about 10 minutes each per day with a fracture cuff over a period of about four (4) weeks.

Each treatment session comprised coupling the cuff up to a compression apparatus for inflating and deflating the cuff and then intermittently pressurising and deflating the cuff to simulate co-contraction of the forearm musculature of the patient. Each compression of the cuff lasted for about 3.5 seconds and each deflation lasted for about 6.5 seconds, giving a pressure application frequency of about 0.1 Hz. Each patient received exactly the same dosage of treatment for the treatment period.

A force applied to the forearm that was generally found not to cause any fracture site pain, while still causing a fairly firm compression of the forearm musculature, is most desirable. This would typically be in the range of about 50 mmHg (6.67 kPa) to 300 mmHg (40 kPa) for a fractured wrist in an adult male. Practitioners will however appreciate that the magnitude or intensity of pressure will vary with the nature of the fracture, the stage of healing and of course the patient's pain threshold. The inventors believe that a compression that stops short of producing any pain at the fracture site will optimise the osteogenic effect, but not adversely interfere with or retard the healing process. Thus the magnitude of pressure applied is governed by the patient's perception of pain at the fracture site.

The rate of healing of each patient's fracture was assessed or measured on a weekly basis. This rate of healing was compared against the rate of healing for a control group of patients who did not receive the treatment set out in this application. The assessment of rate of healing was made by measuring limb function as an indicator of biomechanical strength of the fracture site. The level of function of the fractured arm and associated hand was measured as a percentage of that of the non-fractured arm and associated hand.

Grip strength was measured using a Jamar dynamometer equipment in four (4) positions suited to the forearm. These four measurement positions are as set out below:
grip strength in a sitting position with the elbow fully extended;
pinch strength;
key grip strength; and
isometric supination strength.

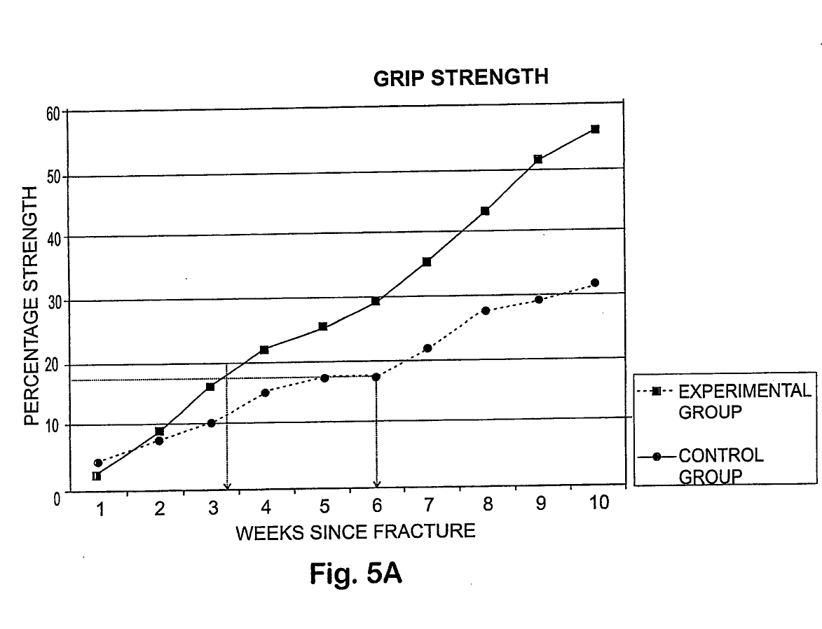

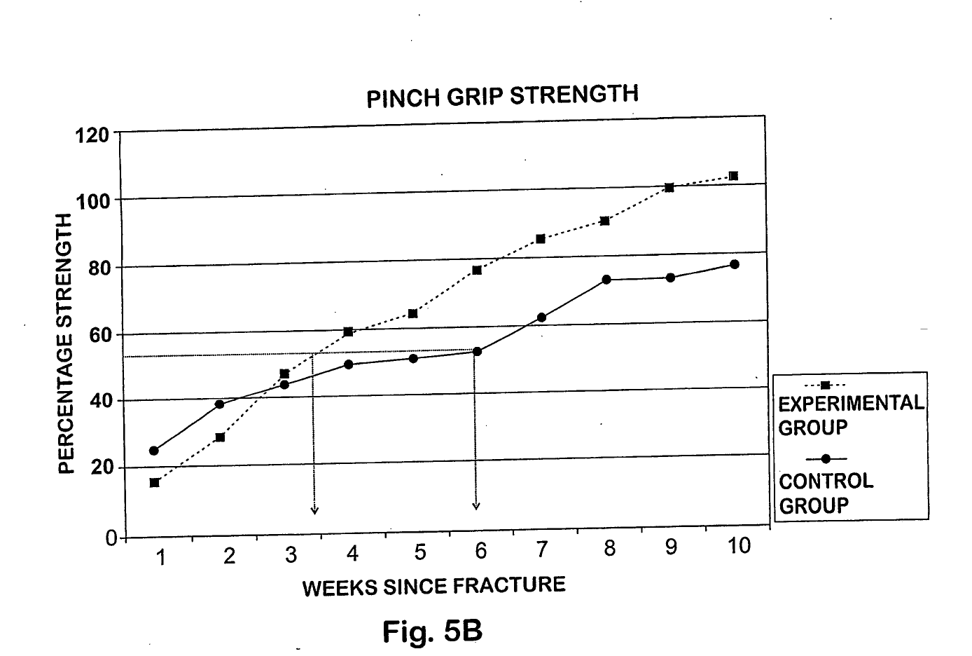

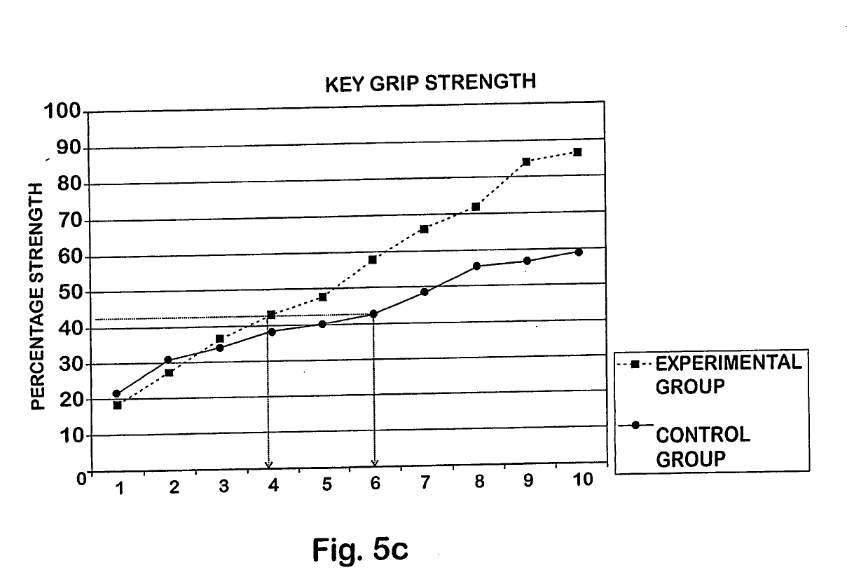

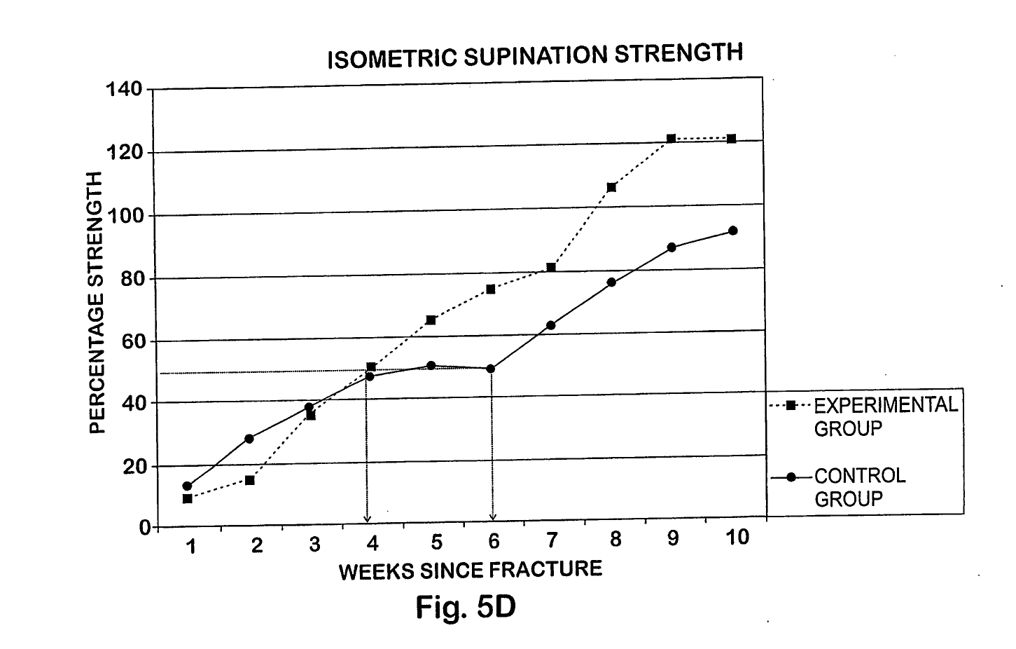

The results of the inventor's experiment for patient strength in each of the above positions are shown in FIGS. 5A to 5D. On average, a patient who was subjected to the treatment of the present invention achieved, in only four (4) weeks, the mean strength after six (6) weeks of patients who were not treated with the invention. The inventor's experimental work demonstrates clearly that healing of fractures of the distal radius occurred at a significantly faster rate when the apparatus and method of the invention was used.

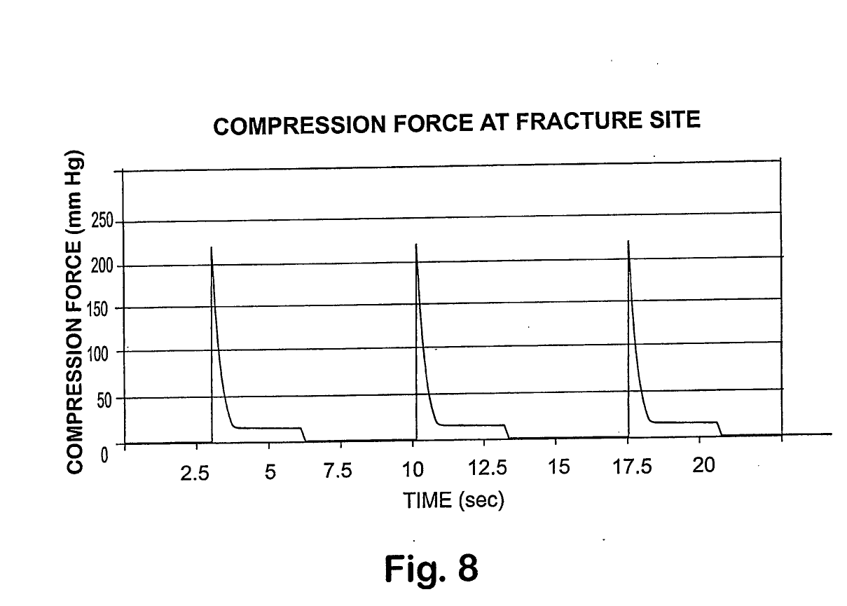

The inventor also conducted experiments to verify that the compression cuff does increase the application of compressive force on the distal radius. A first experiment involved placing a pressure sensor within the radio-carpal joint of a cadaver and then fitting the cuff to the cadaver arm and cyclically inflating and de-deflating the cuff, as described above. The measurements registered by the pressure sensor showed that there was an increase in compressive force in the joint when the cuff was inflated and applied circumferential pressure was applied to the forearm musculature. A transverse fracture of the distal radius shares a similar orientation to the radio-carpal joint. A further experiment was conducted using animal bones, namely from sheep, and employing the cuff of the first embodiment of the present invention. The forces detected across the ends of a fractured distal radius of a sheep undergoing the cyclic pressure treatment using the cuff are depicted in the graph shown at FIG. 8. This demonstrates that the inventor's earlier assumption that the cuff and compression apparatus would also produce these forces in a fracture of the distal radius, is essentially correct.

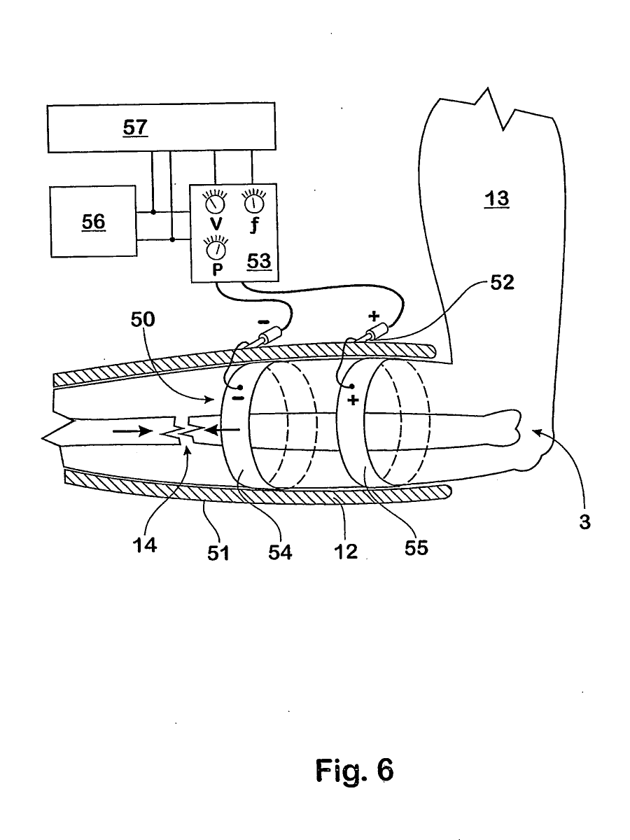

FIG. 6 illustrates another embodiment of the apparatus called the electrical impulse form. The apparatus comprises broadly a signal transmitting member 50 electrically coupled to a signal generator 53.

The member 50 includes two bands 54, 55 that extend fully around the arm 13 of a patient and apply electrical impulse signals directly to the skin of the patient. The electrically active part of the member 50, comprising positive and negative electrodes carried or comprised by respective bands 54, 55, also extend fully around the limb of the patient. The signal transmitting member 50 is received within an immobilisation cast 51 much like the embodiment described above with reference to FIGS. 1 and 2. Each of the bands has an electrical terminal 52 projecting out through an opening in the cast 51 which in use couples the electrodes to the signal generator 53.

In use for each treatment session the generator 53, which is powered by a battery pack 56, is coupled to the signal transmitting member 50. The generator 53 is then switched on to cause intermittent electrical stimulus to be transmitted by means of the bands 54, 56 through the skin to the musculature of the limb, in particular the muscles traversing the fracture 14. The bands are suitably fitted to the limb adjacent the motor end points of the muscles, which are usually found in the proximal third of said muscles. The application of the electrical stimulus causes, in response, contraction of the musculature to which applies longitudinal compression to the fractured bone similar to that achieved with the fracture cuff 15, except that in this embodiment the contraction of the musculature is active rather than passive.

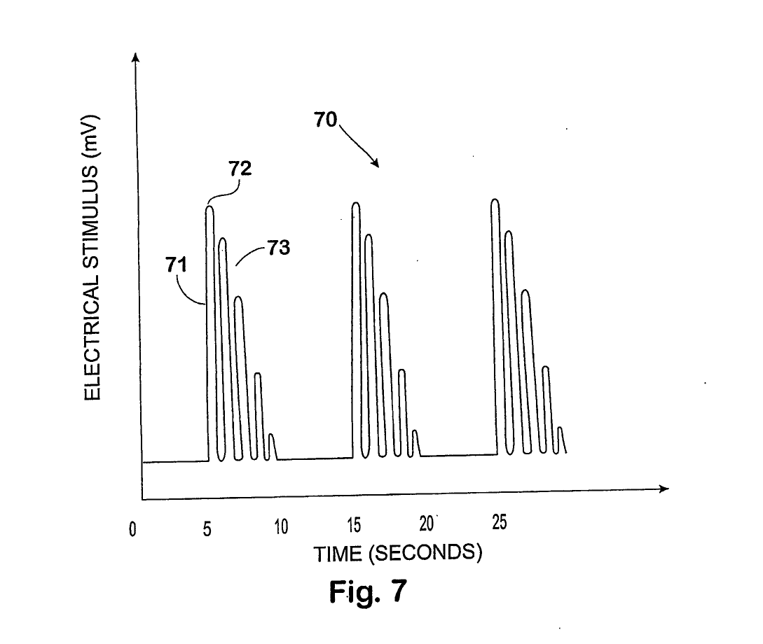

The duration of each treatment session and the frequency of treatment sessions is broadly similar to those described above with reference to FIGS. 1 and 2. However, it will be appreciated from the preferred signal characteristic 70 depicted In FIG. 7, the electrical stimulation signal of this embodiment employs a carrier signal (not to scale in the drawing for reasons of clarity), having a frequency in the range of about 30 Hz to 50 Hz, that is amplitude modulated by the desired impulsive stimulus. The underlying carrier frequency range of about 30 Hz to 50 Hz was chosen in view of the known physiological responses of the local nervous system to high voltage pulsed current.

The impulsive stimulus signal has a relatively fast rise 71, with a short hold at maximum amplitude 72 (of about 0.5 s), followed by a slower decay 73, resulting in a modulated signal having a somewhat saw-tooth shaped outer envelope. The amplitude of the signal may vary in the range from 0 to 500v, with appropriately low current, consistent with safe electro-medical practice. It will be appreciated that the signal generator 53 may be programmable such that any desired characteristic may be selected by a clinician, as required.

The signal generator 53 is suitably provided with adjustments for each of the frequency (f) of the underlying carrier signal, the maximum amplitude of the impulsive signal (V) and the period (P) of the modulating stimulus. Typically the amplitude is adjusted steadily upward from a nominal value, in order that compressions may be applied to the fracture without causing pain or at most moderate discomfort to the patient. As discussed earlier, it will be appreciated that variations in the nature of the fractures, the patients and stage of healing call for different parameter settings.

The electrical impulse form of the invention has the advantage that stimulation of the musculature to actively effect periodic compression of the fracture site assists in the reduction of muscle wastage, which accompanies immobilised limbs. Furthermore the apparatus, when in the form of a portable battery operated signal generator is compact and may be conveniently and discretely used at any time by the patient, has few moving parts and is susceptible to low cost production. A data recorder 57 may also be included for recording the time, date, duration and other details of the treatment. This can assist clinical monitoring of patients that are undertaking self-treatment, in that the recorded data will reveal whether the prescribed treatment regime is adhered to by a patient.

In some circumstances, such as patients prone to epileptic episodes or having metal implants or heat pacemakers, the air pressure cuff will find application.

The essence of the present invention resides in the discovery that active or passive contraction of the musculature associated with the fractured limb causes longitudinal compression of the fracture site and thereby aids in healing the fracture. Further the contraction of the musculature may be induced non-invasively by an external agency.

Without being bound by theory, the applicant believes that mechanical stimulation of the fracture site described above stimulates new bone formation at the fracture site. Desirably the mechanical stimulation should be consistent with normal usage, for example at or below normal walking pace. By contrast the current standard clinical treatment for long bone fractures namely of a rigid immobilisation of the fractured limb substantially decreases if not removes mechanical stimulation of the joint site at a time when the rate of new bone production is required to be at its highest.

The applicant believes it likely that the frequency range, duration and intensity of electrical stimulation or mechanical compression all influence the rate of bone healing in a fracture and therefore applicant envisages increasing the duration or intensity of each electrical stimulation or mechanical compression progressively as the bone heals. As the bone heals it should be able to withstand greater stimuli without the patient suffering fracture site pain or at most moderate discomfort. For example one or more of the frequency, duration or intensity of treatment may be graduated, for example increased, as the bone healing progresses.

Treatment protocols will be developed directed to enhancing and optimising the osteogenic effect without increasing discomfort to the patient in the form of fracture site pain or causing damage to the healing fracture site. For example the mechanical pressure or electrical stimulus might be increased every few days or every week up to the point that falls just short of producing fracture site pain. It is expected that the point just short of pain will be different for different patients and different fractures.

An advantage of the treatment described above with reference to the drawings, is that the treatment has been found to enhance the rate of healing of the fracture. The increase in rate of recovery shown in FIGS. 5A to 5D is very significant, since bone strength is increased at least 25% in comparison with conventional treatment and therefore would shorten the typical recovery time by at least a couple of weeks. A further advantage is that it is non-invasive and does not expose the patient to risk of infection or other injury. Further the treatment does not cause discomfort or pain to the patient.

A yet further advantage is that, upon provision of the necessary apparatus, treatment can be carried out by a patient in their home. The treatment does not need to be administered by a health care professional, since it is substantially automated. This makes it more user-friendly and suited to the modern lifestyle where many people have difficulty finding time to visit clinics.

A still further advantage of the apparatus described above with reference to the drawings is that it is relatively simple, particularly in the cuff or sleeve embodiment. This helps to make the technology affordable and also predictable and reliable in operation. A yet further advantage of this simplicity is that it should help in obtaining acceptance of the technology by health care providers.

It will of course be realised that the above has been given only by way of illustrative example of the invention and that all such modifications and variations thereto as would be apparent to persons skilled in the art are deemed to fall within the broad scope and ambit of the invention as is herein set forth in the following claims.

1. A method for treating a long bone fracture in a limb of an animal or a human, the method comprising the steps of:
non-invasively and intermittently inducing a shortening in the axial length of skeletal musculature surrounding the fractured long bone in said limb;
whereby the skeletal musculature intermittently applies a longitudinal compressive force to the fractured bone.

2. The method of claim 1 wherein the step of inducing the shortening in the axial length of skeletal musculature is achieved by applying a force or stimulus radially to the outside of the limb that is transmitted to the surrounding muscles.

3. The method of claim 2 wherein the radial force or stimulus is applied dynamically in that it varies as a function of time.

4. The method of claim 2 wherein the radial force or stimulus is applied at regular intervals.

5. The method of claim 2 wherein the radial force or stimulus is applied to the outside of the limb in a balanced fashion.

6. The method of claim 2 wherein the radial force or stimulus is applied substantially fully around the circumference of the limb.

7. The method of claim 2 wherein the radial force or stimulus is applied by a transmitting member that extends around the outside of the limb.

8. The method of claim 7 wherein the transmitting member is displaced along the limb so as to be longitudinally spaced from the fracture.

9. The method of claim 2 wherein the radial force or stimulus has a magnitude below that which causes the patient fracture site pain.

10. The method of claim 9 wherein the radial force or stimulus has a magnitude just below that which causes fracture site pain or at most moderate discomfort.

11. The method of claim 7 wherein the transmitting member is a mechanical force application member which applies pressure to the outer surface of the limb.

12. The method of claim 7 wherein the transmitting member is an electrical signal transmitter which applies electrical stimulus to the outer surface of the limb.

13. The method of claim 11 wherein the duration of application of the force or stimulus is between about 2 to 7 seconds.

14. A method for treating a long bone fracture in a limb of an animal or a human, the method comprising the steps of:
non-invasively and intermittently inducing a shortening in the axial length of skeletal musculature surrounding the fractured long bone in said limb by applying a force or stimulus radially to the outside of the limb that is transmitted to the surrounding muscles;
whereby the skeletal musculature intermittently applies a longitudinal compressive force to the fractured bone to effect a plurality of compressions of the fractured bone over a predetermined period of time in a treatment session.

15. The method of claim 14 wherein the treatment session includes applying at least 20 compressions to the fracture.

16. The method of claim 14 wherein the treatment session comprises from about 40 to 70 compressions.

17. The method of claim 14 including at least one treatment session each day.

18. The method of claim 17 including two treatment sessions each day spaced 8 to 16 hours apart.

19. The method of either claim 17 or claim 18 wherein the treatment sessions are conducted for at least 10 days.

20. The method of claim 17 wherein the treatment sessions commence within about 5 to 10 days post fracture.

21. An apparatus for treating a long bone fracture in the limb of an animal or human, said apparatus comprising:
non-invasive means for intermittently inducing a shortening in the axial length of the skeletal musculature surrounding the long bone, whereby a compressive force may be intermittently applied to the fractured bone.

22. The apparatus of claim 21 wherein the means for inducing a shortening of the skeletal musculature comprises a force application member for applying a force radially to the limb that is transmitted to the surrounding muscles.

23. The apparatus of claim 22 wherein the force application member is adapted to apply the radial force dynamically, in that the force may be varied as a function of time.

24. The apparatus of claim 22 wherein the radial force may be selectively applied at regular intervals in the range from 0.05 Hz to 2 Hz.

25. The apparatus of claim 22 wherein the radial force is applied to the outside of the limb in a balanced fashion.

26. The apparatus of claim 22 wherein the radial force is applied to the limb by a force application member extending fully circumferentially around the limb.

27. The apparatus of claim 22 wherein the force application member is adapted to apply the radial force for a short period and then released the limb, and thereafter this cycle of force application and release may be repeated.

28. The apparatus of claim 22 wherein the force application member is a sleeve or cuff that passes over the limb of a patient, which sleeve or cuff is expandable and contractible on demand to apply said force to the limb of the patient.

29. The apparatus of claim 28 wherein the sleeve or cuff has a generally ring or toroidal shaped body that is adapted to retain a fluid in said body.

30. The apparatus of claim 28 wherein the force application member may be inflated and deflated on demand by the forced introduction and subsequent release of air through a valve and/or fluid a coupling.

31. The apparatus of claim 21 further comprising an immobilisation member for immobilising the fractured limb of the patient.

32. The apparatus of claim 31 wherein the immobilisation member comprises a relatively rigid cast of plastic or plaster for supporting the limb.

33. The apparatus of claim 31 wherein the force application member is positioned under the immobilisation member, preferably sandwiched between the immobilisation member and the skin of the patient.

34. The apparatus of claim 31 wherein the force application member is positioned adjacent the immobilisation member, preferably abutting the proximal end of said immobilisation member.

35. The apparatus of claim 31 wherein the valve or fluid coupling projects through the immobilisation member and provides one form of attachment of the cuff to the cast.

36. The apparatus of claim 31 may further including a means for selectively inflating the cuff with compressed air to a predetermined pressure for a predetermined time and then deflating the cuff by releasing said pressure.

37. The apparatus of claim 21 adapted for delivering the treatment of a method for treating a long bone fracture in a limb of an animal or a human, the method comprising the steps of:
non-invasively and intermittently inducing a shortening in the axial length of skeletal musculature surrounding the fractured long bone in said limb:
whereby the skeletal musculature intermittently applies a longitudinal compressive force to the fractured bone.

38. The apparatus of claim 21 wherein the means for inducing a shortening of the skeletal musculature comprises an electrical signal transmitter for generating an electrical impulse for stimulating the skeletal musculature and thereby causing it to contract and shorten to apply longitudinal compression to the bone.

39. The apparatus of claim 38 wherein the electrical signal transmitter comprises a signal transmitting member that is placed in contact with the outer surface of the limb of the patient that applies an electrical stimulus to the limb of the patient.

40. The apparatus of claim 38 further comprising electrodes placed on the skin of a user, which electrodes are arranged to pass fully circumferentially around the arm of a user for application of said stimulus so as to induce contraction of the muscles in the limb traversing the fracture.

41. The apparatus of claim 38 wherein the signal generator is of the battery powered portable type.

42. The apparatus of claim 38 wherein the signal generator is programmable.

43. The apparatus of claim 38 wherein the signal generator is associated with a data recorder for recording details of the electrical stimulus applied to the musculature.

44. The apparatus of claim 38 wherein the electrical stimulus comprises an underlying carrier signal that is amplitude modulated by an impulse signal.

45. The apparatus of claim 44 wherein the carrier signal has a frequency in the range from about 30 Hz to 50 Hz.

46. The apparatus of either claim 44 or claim 45 wherein the impulse signal has a frequency in the range from 0.05 to 2 Hz.

47. A compression apparatus for inflating and deflating a cuff or sleeve of an apparatus for treating a long bone fracture in the limb of an animal, the apparatus adapted for selectively pressurising and inflating the cuff by supplying compressed air to the cuff and subsequently deflating the cuff, such that the cuff may be intermittently inflated and deflated; said compression apparatus comprising:
inflation device for inflating the cuff or sleeve such that the cuff applies pressure radially inwardly to the limb and, after a desired compression of the fracture has been achieved;
pressure relief device for relieving that pressure from the limb by deflating the cuff; and
timer for controlling respective durations of inflation and deflation.

48. The compression apparatus of claim 7 further comprises:
a closed tank container for supplying pressurized fluid;
a valve having an air inlet port operatively coupled to the pressurised fluid supply and a vent port for venting fluid to the atmosphere in parallel with the inlet port;
a cuff coupling for operatively coupling the valve to the cuff;
the valve having a switch for switching either the fluid inlet port or the vent port to the cuff at any one time, whereby only one of the fluid inlet or the vent port can be coupled to the cuff or sleeve, whereby the apparatus is either in compression mode or in deflation mode at any one time.

49. The compression apparatus of claim 48 wherein the valve comprises a solenoid actuated two-way valve and the pressurised fluid is compressed air.

50. The compression apparatus of claim 47 wherein the timer means further includes timer setting device for setting the duration for which the cuff is inflated and also the duration for which the cuff is deflated. 51. The method of claim 12, wherein the duration of application of the force or stimulus is between about 2 to 7 seconds.
